(19) United States
(12) Patent Application Publication
(10) Pub. No.: US 2022/0016661 A1
(43) Pub. Date: Jan. 20, 2022

(54) CHANNELED POROUS FILTER FOR CONTROLLED-RELEASE OF FLUIDS

(71) Applicant: Acryl8, LLC, Coopersburg, PA (US)

(72) Inventors: Jim Tobin, Coopersburg, PA (US); Eric Rohde, Bloomington, IL (US); Jeff Burk, Bloomington, IL (US)

(21) Appl. No.: 17/376,975

(22) Filed: Jul. 15, 2021

Related U.S. Application Data
(60) Provisional application No. 63/052,223, filed on Jul. 15, 2020.

(51) Int. Cl. B05C 1/00; B05C 11/10

(52) U.S. Cl. CPC B05C 1/00; A61B 17/00491; B05C 11/10

(57) ABSTRACT

A porous filter includes a body including a first body portion and a second body portion, wherein the body includes an outer surface and a length, and wherein the first body portion has a cylindrical shape. An inner channel is formed along the length of the body and includes an open channel end on the outer surface on the first body portion and an internal channel end within the body.

### CROSS-REFERENCE TO RELATED APPLICATIONS

This application claims the benefit of priority to U.S. Provisional Application No. 63/052,223 filed Jul. 15, 2020, the entirety of which is incorporated herein by reference.

### BACKGROUND OF THE INVENTION

The present subject matter relates generally to porous filter used in devices for dispensing fluids. More specifically, the present invention relates to a channeled porous filter that allows for the controlled release of fluid through the filter.

Topical skin adhesives are commonly applied to skin to close wounds, such as lacerations, incisions, and wounds on the face and other curved surfaces of the body. While Steri-strips and Band-Aids® are appropriate for minor cuts and scrapes, more severe injuries may warrant stitches or staples. Skin glue can be used in conjunction with stitches or staples, or on its own if the site of the injury is not conducive to bandages, such as near the eye.

Conventional skin glues are sold in a single-use applicator for applying the glue directly onto the skin. Skin glue applicators typically include an ampoule for storing the adhesive and fitted with a tip. During use, pressure is applied to the ampoule which allows the adhesive to flow through the tip. In one type of applicator, the tip comprises a porous material that controls the flow of adhesive as well as serving other uses.

In applicators that use a porous filter tip, the combination of the viscosity of the fluid and the shape and porosity of the tip control the direction and flow of the fluid. Alternative filter designs have included various material to increase or decrease porosity. These designs resulted in flow rates which were not optimized in that they either allowed flow rates which were too fast (fluid flowed faster than the desired rate) or too slow (fluid required a great deal of pressure to pass through the filter) for certain applications.

Other alternative designs have included a decrease in the length of the filter to reduce the flow resistance of the material. Unfortunately, these designs have had various shortcomings including fluids tending to flow toward the outer edges of the filter rather along a path of least resistance rather than through the filter material.

Accordingly, there is a need for a porous filter that allows for fluids to flow through easily and directionally targeted as described herein.

### SUMMARY

To meet the needs noted above and others, the present disclosure provides a porous filter including an inner channel configured to direct flow. The inner channel design may include a single centrally-located channel, a plurality of channels, or a main channel with a plurality of secondary channels connected thereto. By providing a channeled porous filter, fluid flows directionally through the porous filter material and allows for easier expression of fluid. While the porous channeled filter is described with reference to a medical-grade skin glue applicator, the porous filter may be used in a wide variety of settings, from tubing systems to control flowrates and capture particulate matter in an industrial setting to medical applications such as catheters, dialysis systems, and others.

In the embodiments illustrated herein, the porous filter is attached to a container or ampoule of a fluid dispensing assembly through a filter collar. The filter collar is a body having a first cavity for attaching to a nozzle of the container and a second cavity for receiving the porous filter. A bore extending through a central axis of the filter collar provides a throughway for fluid to flow from the container into the porous filter.

In some embodiments, the first cavity of the filter collar has internal threading that engages with external threading on the nozzle of the container. Each of the first and second cavities are defined by first and second side walls, respectively, and first and second end surfaces spanning the respective side walls. First and second tubing portions extend from the first and second end surfaces, respectively, of the first and second cavities, respectively, so that the bore extends from directly adjacent to the nozzle within the first cavity to the interior of the porous filter in the second cavity during use.

The porous filter includes a cylindrical portion that is received by the second cavity of the filter collar and a hemispherical portion that provides an application surface. The inner channel is provided along the central axis of the porous filter. An open end of the inner channel is provided on an end surface of the cylindrical portion, and an inner channel end is formed within the porous filter spaced from an outer surface thereof such that the fluid flows into cavities in the porous material as it moves out of the inner channel and through the porous filter.

During use, the user expresses fluid out of the container or ampoule in the direction of the porous filter. The interior channel(s) or cavity(ies) provide additional surface area on the porous filter through which the fluid may enter. Compression on the ampoule provides pressure to force the fluid through the channel(s) and out of the end of the porous filter.

In light of the disclosure set forth herein, and without limiting the disclosure in any way, in a first aspect, which may be combined with any other aspect or portion thereof described herein, a porous filter includes a body including a first body portion and a second body portion. The body includes an outer surface and a length, and the first body portion has a cylindrical shape. An inner channel is formed along the length of the body and includes an open channel end on the outer surface on the first body portion and an internal channel end within the body.

In a second aspect, which may be combined with any other aspect or portion thereof described herein, the first and second body portions are continuous. In some embodiments, the second body portion has a hemispherical shape.

In a third aspect, which may be combined with any other aspect or portion thereof described herein, the inner channel extends between about 5% to about 95% of the length of the body. In some example embodiments, the internal channel end is within the first body portion.

In a fourth aspect, which may be combined with any other aspect or portion thereof described herein, the inner channel has a first inner channel portion and a second inner channel portion. The first inner channel portion has a cylindrical shape, and the second inner channel portion has a hemispherical shape.

In a fifth aspect, which may be combined with any other aspect or portion thereof described herein, the porous filter includes a plurality of inner channels formed along the length of the body. In one embodiment, the plurality of channels includes channels having identical shapes. In other embodiments, the plurality of channels includes a main channel portion and at least two channel portions extending from the main channel portion.

In a sixth aspect, which may be combined with any other aspect or portion thereof described herein, a fluid dispensing assembly includes a container including a nozzle, a filter collar, and a porous filter. The filter collar includes a collar body having a length extending between a first end and a second end, a first cavity at the first end, a second cavity at the second end, and a bore extending between the first cavity and the second cavity. The porous filter includes a body including a first body portion and a second body portion, wherein the body includes an outer surface and a length and wherein the first body portion has a cylindrical shape. The porous filter also includes an inner channel formed along the length of the body and including an open channel end on the outer surface on the first body portion and an internal channel end within the body. The porous filter is received within the second cavity of the filter collar such that the inner channel is aligned with the bore of the filter collar.

In a seventh aspect, any of the features, functionality, and alternatives described in connection with any one or more of FIGS. 1 to 21 may be combined with any of the features, functionality, and alternatives described in connection with any other of FIGS. 1 to 21.

An object of the present invention is to increase directional flow rate of the adhesive through the porous filter and reduce the force to express the fluid through the filter.

An advantage of the present invention is to provide a simple solution for increasing the efficiency of the design.

Additional features and advantages are described herein, and will be apparent from the following Detailed Description and figures.

### BRIEF DESCRIPTION OF THE DRAWINGS

FIG. 1 is a perspective view of a fluid dispensing assembly of the present application.

FIG. 2 is a side elevational view of the fluid dispensing assembly of FIG. 1.

FIG. 3 is a cross sectional view taken along lines A-A of FIG. 2 of the fluid dispensing assembly of FIG. 1.

FIG. 4 is an expanded view of the filter collar attached to the container of the fluid dispensing assembly of FIG. 1.

FIG. 5 is an expanded view of the porous filter attached to the filter collar of the fluid dispensing assembly of FIG. 1.

FIGS. 6 and 7 are plan and bottom plan views of the fluid dispensing assembly of FIG. 1.

FIGS. 8 and 9 are perspective views from above and from below, respectively, of the filter collar of the fluid dispensing assembly of FIG. 1.

FIG. 10 is a cross sectional view taken along lines A-A of FIG. 2 of the filter collar of FIGS. 8 and 9.

FIGS. 11 and 12 are plan and bottom plan views, respectively, of the filter collar of FIGS. 8 and 9.

FIGS. 13 and 14 are perspective and plan views, respectively, of an exemplary porous filter of the fluid dispensing assembly of FIG. 1.

FIG. 15 is a cross-sectional view taken along lines B-B of the porous filter of FIG. 14.

FIGS. 16 and 17 are perspective and plan views, respectively, of an alternative embodiment of the porous filter of the fluid dispensing assembly of FIG. 1.

FIG. 18 is a cross-sectional view taken along lines C-C of the porous filter of FIG. 17.

FIGS. 19 and 20 are perspective and plan views, respectively, of a further alternative embodiment of the porous filter of the fluid dispensing assembly of FIG. 1.

FIG. 21 is a cross-sectional view taken along lines D-D of the porous filter of FIG. 19.

### DETAILED DESCRIPTION

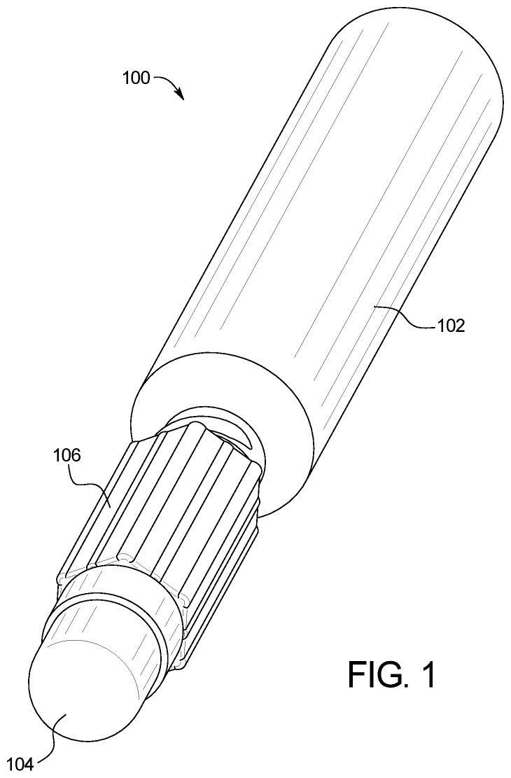

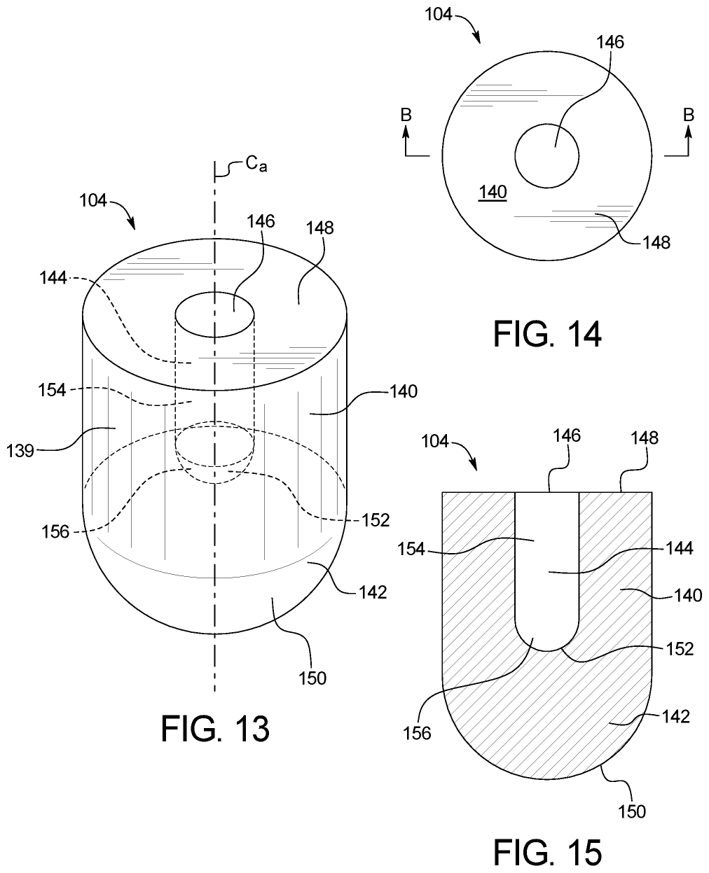

FIGS. 1-15 illustrate an example embodiment of a fluid dispensing assembly 100, including a container 102 containing a fluid such as an adhesive material, a porous filter 104 used to apply the fluid to a user's body, and a filter collar 106 into which the porous filter 104 is secured. Seen best in FIG. 13, the porous filter 104 includes an inner channel 144 that receives fluid from the container 102 via the filter collar 106 and directs the fluid into cavities throughout the porous filter material, improving the directional flow of fluid through the porous filter material and of expression of fluid.

While the illustrated embodiment features the porous filter 104 as part of a specific fluid dispensing assembly 100 for skin adhesive as described herein, the porous filter 104 is not limited to such application. The porous filter 104 may be used in a wide variety of applications, ranging from medical uses to industrial applications. In the medical industry, the porous filter 104 may be a component in larger systems such as catheters or dialysis systems. In an industrial setting, the porous filter 104 can be used in a number of settings, such as a tubing system with a porous filter to control flowrates and/or capture certain particulates.

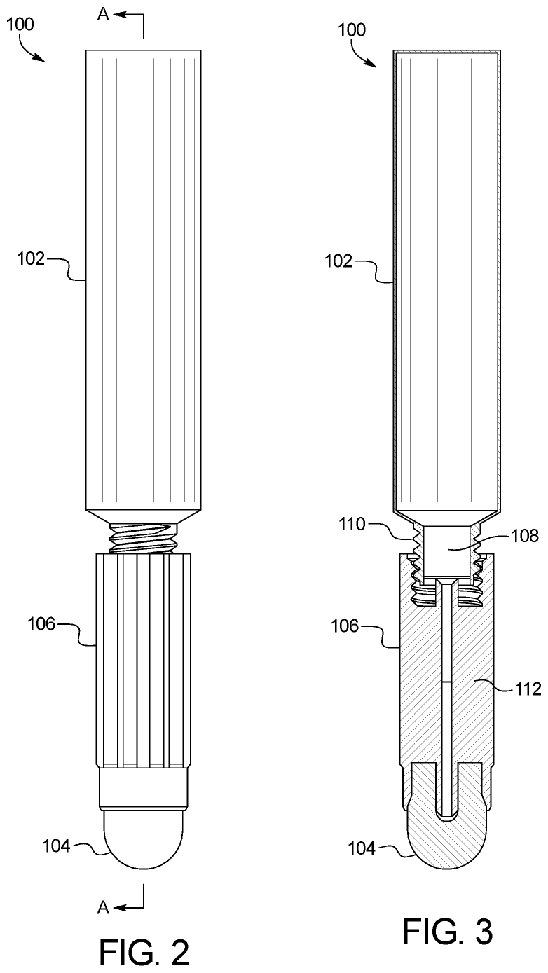

Referring to the embodiment illustrated in FIGS. 1 and 2, the fluid dispensing assembly 100 includes a container 102 such as an aluminum tube container containing an inactive adhesive material or an inner glass ampoule containing an inactive adhesive material housed within a plastic tube. During use, pressure is applied to the container 102 and adhesive material is expressed through a nozzle 108 of the container 102 through the filter collar 106 and into the porous filter 104. The porous filter 104 is impregnated with an actuating agent which activates the adhesive as it flows through the filter 104.

In the container embodiment including the inner glass ampoule within a plastic tube, pressure applied to the plastic tube causes the glass ampoule to break and adhesive material to be expressed out of the nozzle of the plastic tube. Where an aluminum tube container is used, the nozzle may include a membrane sealing that requires puncturing prior to expressing the adhesive material therefrom. Other embodiments of the fluid dispensing assembly may utilize other suitable containers.

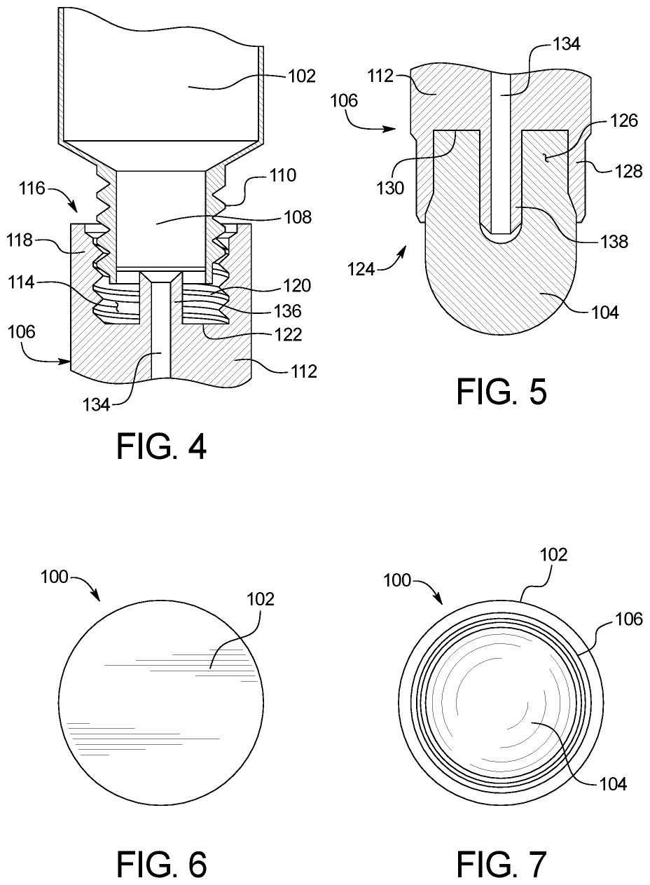

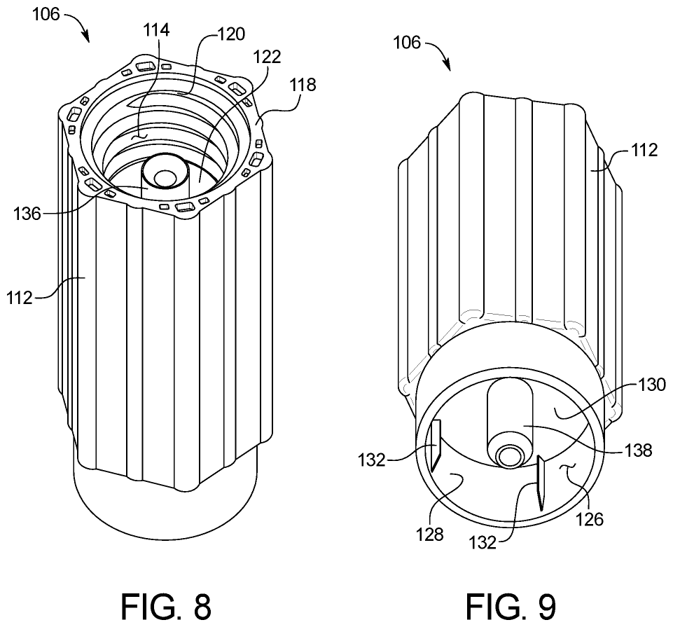

As shown in FIGS. 3-5, the filter collar 106 provides a fluid connection between the container 102 and the porous filter 104. As shown in FIGS. 3 and 4, the nozzle 108 of the container 102 has external threading 110 that engages the filter collar 106. More specifically, the filter collar 106 includes a collar body 112 with a first cavity 114 at a first end 116 that receives the nozzle 108 of the container 102. Referring to FIGS. 8, 10, and 11, the first cavity 114 of the filter collar 106 includes a side surface 118 with internal threading 120 that engages the external threading 110 on the nozzle 108. An end surface 122 of the first cavity 114 spans the side surface 118. While threading is provided in the illustrated embodiment to secure the connection of the components, other means of attaching such as a press fit connection may also or alternatively be used as desired. Similarly, the shape and dimensions of the first cavity can be modified to accommodate container nozzles having different sizes and shapes.

At a second end 124 of the body 112 shown in FIGS. 5, 9, 10, and 12, a second cavity 126 is configured to receive and securely hold the porous filter 104 in place. In the illustrated embodiment, a side surface 128 of the second cavity 126 has a cylindrical shape that complements the outer shape of the porous filter 104. An end surface 130 of the second cavity 126 spans the side surface. In other embodiments, the size and shape of the second cavity 126 may be modified to accommodate porous filters having different sizes and shapes as described herein.

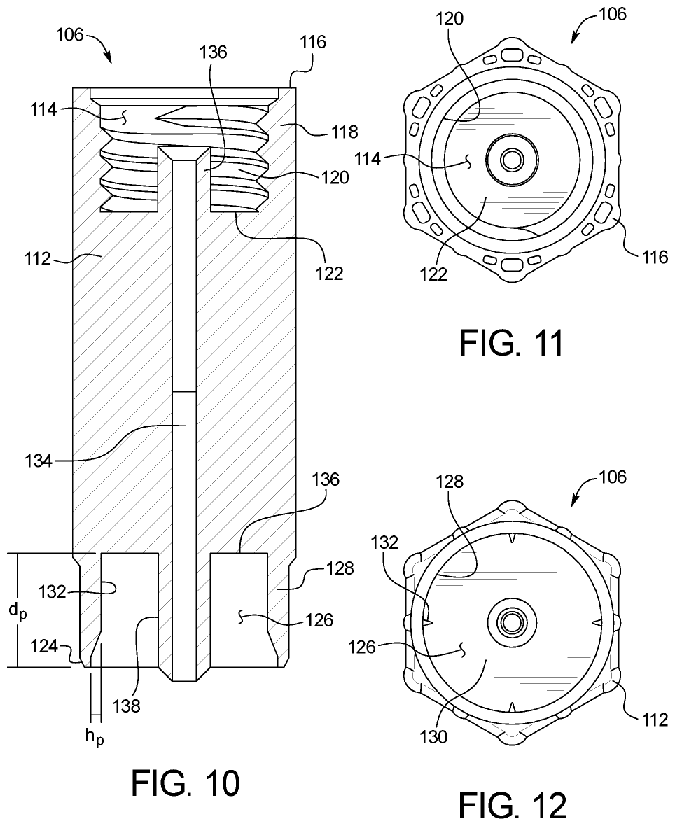

Referring to FIGS. 9 and 10, one or more projections 132 may extend inwardly from the side surface 128, with each projection 132 having a height hₚ measured from the side surface 128 that tapers with distance dₚ from the end surface 130 of the second cavity 126. In the illustrated embodiment, the porous filter 104 is held in place through a press-fit connection. In other embodiments, mechanical means, ridged or lipped surfaces, adhesives, or other components and materials may be used as desired.

Referring to FIG. 10, a bore 134 provides a throughway within the body 112 between the first cavity 114 at the first end 116 and the second cavity 126 at the second end 124 for the adhesive material or other fluid to move from the container 102 into the porous filter 104 (see FIGS. 3-5). In one example embodiment, the bore 134 has a width of at least 0.031 in, although any dimension may be used for desired flow characteristics or as needed per manufacturing needs.

A first tubular portion 136 defining a portion of the bore 134 extends from the end surface 122 of and into the first cavity 114 at the first end 116 of the body 112, seen best in FIGS. 4, 8, 10, and 11. During use as shown in FIG. 4, the first tubular portion 136 is positioned adjacent and/or extends into the nozzle 108 when the filter collar 106 is threaded onto the nozzle 108 of the container 102. The tubular portion 136 and the threaded connection 110, 120 ensure that fluid passes from the nozzle 108 into the bore 134.

Seen best in FIGS. 5, 9, 10, and 12, a second tubular portion 138 defining another portion of the bore 134 extends from the end surface 130 of and into the second cavity 126 at the second end 124 of the body 112. During use as shown in FIG. 5, the second tubular portion 138 extends into the porous filter 104, ensuring that fluid passes from the bore 134 and is directed to the inner channel 144 of the porous filter 104 as described below.

Outer ends of the first and second tubular portions 136, 168 may have chamfered or angled edges to further direct flow into and out of the bore 134. An outer surface of the filter collar 106 may be faceted and/or textured for easy threading onto and off of the container 102.

Referring to FIG. 5, the porous filter 104 is positioned within the second cavity 126 of the filter collar 106. In the embodiment illustrated in FIGS. 13-15, the porous filter 104 has a body 139 including a first body portion 140 with a cylindrical shape and a second body portion 142 with a hemispherical shape. At least part of the first body portion 140 is sized to fit snugly within the second cavity 126 of the filter collar 106, as shown in FIG. 5, while the second body portion 142 extends from the filter collar 106 and provides a surface that contacts the user's skin when applying the skin adhesive.

In the illustrated example, a width of the first body portion 140 is dimensioned to be slightly greater than a width of the second cavity 126 of the filter collar 106 so that the porous filter 104 is retained in place on the filter collar 106 as fluid is expressed through the filter 104. In other embodiments, the size and shape of the porous filter 104 may vary from the illustrated embodiments and further may correspond to another sized and shaped cavity of the filter collar 106.

Seen best in FIGS. 13-15, the inner channel 144 of the porous filter 104 extends along a central axis Cₐ of the body. An open channel end 146 of the inner channel 144 on an end surface 148 of the porous filter 104 receives the second tubular portion 138 of the filter collar 106 during use so that fluid expressed from the container 102 through the filter collar 106 is directed into the inner channel 146 of the porous filter 104 and permeates through the porous filter 104 to an outer surface thereof 150.

The inner channel 144 of the porous filter 104 terminates at an internal channel end 152 within the porous filter 104 as shown in FIG. 15. The internal channel end 152 is spaced from the outer surface 150 of the porous filter 104 such that the fluid flows into cavities in the porous material as it moves through the porous filter 104. In some embodiments, the inner channel extends between about 5% to about 95% of the length of the body

During use, the inner channel 146 fills with the adhesive material or fluid as it is being expressed out of the container 102, and increases the directional flowrate and ease of expression of the adhesive through the porous filter. In other embodiments, the body of the porous filter may include a plurality of cavities in addition to the inner channel.

In the embodiment illustrated in FIGS. 13-15, the channel 144 has a shape that mirrors the shape of the porous filter 104, with the inner channel 144 including a cylindrical portion 154 and a hemispherical portion 156. In other embodiments, the inner channel 144 may have a different shape and size as desired. For example, the inner channel 144 may have an oblong or planar shape along the end surface 148 of the porous filter 104. The hemispherical shape 156 may have a radius of curvature that is identical to or different than the radius of curvature of the hemispherical portion 142.

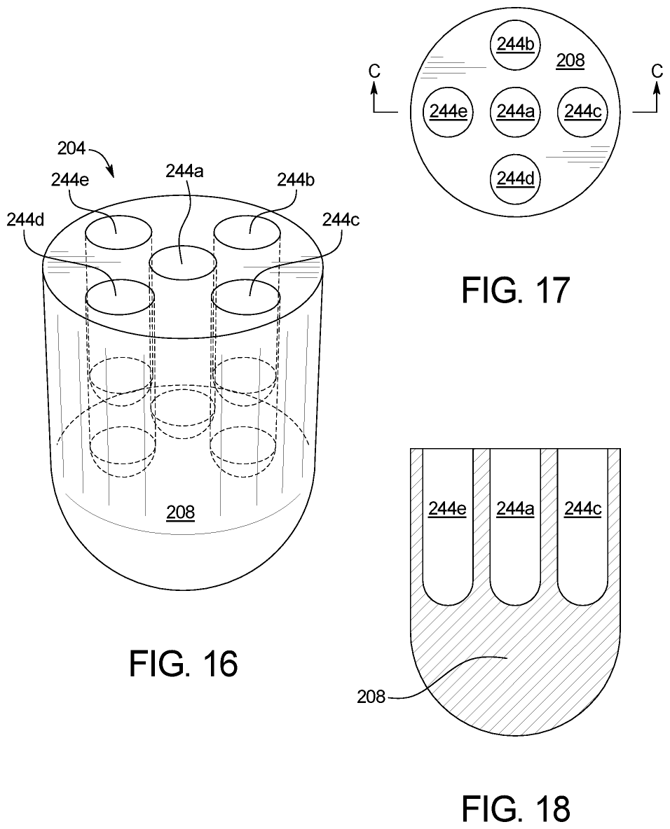

In a further embodiment illustrated in FIGS. 16-18, an alternative porous filter 204 may include five channels 244a-244e, each channel 244 having a shape that corresponds to the shape of the porous filter 104. A first channel 244a is centered along a central axis Cₐ of the porous filter 104, and the second through fifth channels 244b-244e are positioned about the first channel 244a with equal spacing. In other embodiments, the spacing may be varied.

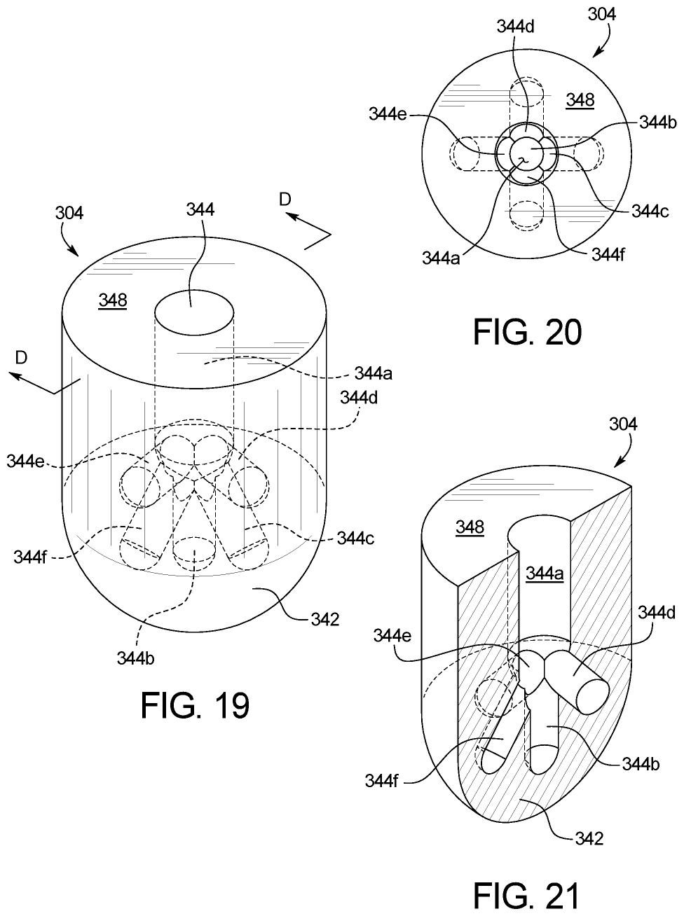

In another embodiment of an alternative porous filter 304 illustrated in FIGS. 19-21, the inner channel 344 has a shape that differs from the shape of the porous filter 304. The inner channel 344 as shown in FIGS. 19 and 21 includes a main channel portion 344a having a cylindrical shape and being centrally located on the end surface 348 of the porous filter 304, similar to the inner channel 144 of the porous filter 104 shown in FIG. 13. In the porous filter 304 of FIGS. 19-21, the inner channel 344 includes a plurality of lower channel portions 344b-344f radiating from the main channel portion 344a into the hemispherical portion 342 of the porous filter 304. The number, dimensions, and positioning of the secondary channels may be modified as desired.

In other embodiments, the porous filter 104 may include any number of inner channels 144, such as two, four, seven, etc. For example, the inner channel 144 may have a diameter to height ratio that differs from the diameter to height ratio of the illustrated porous filters 104, 204, 304. The channel 144 may have a planar or oblong, rather than a cylindrical, shape. For example, the inner channel 144 may have a FIG. 8 shape in plan view. A plurality of channels 144 may extend from the end surface 148 of the porous filter 104 at an angle. Any combination of various design aspects may be used as desired.

In one embodiment, the adhesive may comprise a cyanoacrylate material, and the porous filter is impregnated with a chemical actuator that activates the cyanoacrylate as it passes through the porous filter, causing the cyanoacrylate material to polymerize for use as skin adhesive or medical-grade super glue. Other adhesive materials, fluids, and/or chemical actuators may be used as desired. In still further embodiments, the fluid may not be an adhesive, but may be a pharmaceutical agent, an oil, or water-based fluid with particulate matter.

The porous filter 104 of the present invention may comprise any suitable porous material depending on the type and properties of the fluid being expressed through the porous filter. Where the fluid is a cyanoacrylate material, the porous filter may comprise porous polyethylene. The desired density allows for the expression of fluids with relatively high viscosity indices, although the preferred ranges may vary depending on the fluids flowing through.

The channeled porous filter 104 may be used with a wide range of fluids for controlled-release dispensing. Of course, variations on those the embodiments described herein will become apparent to those of ordinary skill in the art upon reading the foregoing description. Those of ordinary skill in the art are expected to employ such variations as appropriate, and embodiments described herein are to be practiced otherwise than specifically described herein. Accordingly, embodiments herein include all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

Further, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A porous filter comprising:
a body including a first body portion and a second body portion, wherein the body includes an outer surface and a length, and wherein the first body portion has a cylindrical shape;
wherein an inner channel is formed along the length of the body and includes an open channel end on the outer surface on the first body portion and an internal channel end within the body.

2. The porous filter of claim 1, wherein the first and second body portions are continuous.

3. The porous filter of claim 2, wherein the second body portion has a hemispherical shape.

4. The porous filter of claim 1, wherein the inner channel extends between about 5% to about 95% of the length of the body.

5. The filter of claim 1, wherein the internal channel end is within the first body portion.

6. The porous filter of claim 1, wherein the inner channel has a first inner channel portion and a second inner channel portion, wherein the first inner channel portion has a cylindrical shape, and wherein the second inner channel portion has a hemispherical shape.

7. The porous filter of claim 1, further comprising a plurality of inner channels formed along the length of the body.

8. The porous filter of claim 8, wherein the plurality of channels includes channels having identical shapes.

9. The porous filter of claim 8, wherein the plurality of channels includes a main channel portion and at least two channel portions extending from the main channel portion.

10. The porous filter of claim 1, wherein the body comprises a porous material.
